(19) United States
(12) Patent Application Publication
(10) Pub. No.: US 2023/0364305 A1
(43) Pub. Date: Nov. 16, 2023

(54) INJECTABLE CALCIUM PHOSPHATE-BASED BONE GRAFT COMPOSITION HAVING HIGH ELASTICITY AND PREPARATION METHOD THEREOF

(71) Applicant: CG BIO CO., LTD., Gyeonggi-do (KR)

(72) Inventors: Hyunseung RYU, Gyeonggi-do (KR); Jun-Hyuk SEO, Gyeonggi-do (KR); Hyochul JUNG, Seoul (KR); Miyoung RYU, Gyeonggi-do (KR); Ji-hye LEE, Seoul (KR); Hyunjung PARK, Gyeonggi-do (KR)

(73) Assignee: CG BIO CO., LTD., Gyeonggi-do (KR)

(21) Appl. No.: 18/028,650

(22) PCT Filed: Jan. 27, 2021
(86) PCT No.: PCT/KR2021/001110

§ 371 Date: Mar. 27, 2023

(30) Foreign Application Priority Data
Sep. 29, 2020 (KR) 10-2020-0127278

(51) Int. Cl. A61L 27/52; A61L 27/36; A61L 27/32; A61L 27/22; A61L 27/56

(52) U.S. Cl. CPC A61L 27/52; A61L 27/365; A61L 27/32; A61L 27/227; A61L 27/56; A61L 2300/252; A61L 2430/02; A61L 2400/06

(57) ABSTRACT

Provided are a bone graft composition and a preparation method thereof, and more particularly, a bone graft composition provided in the form of a putty formulation by mixing calcium phosphate compound particles with hydrogel, having excellent physical properties, which is easy to inject, and which maintains its structure even in an in vivo environment after implantation, thereby enabling sustained release of a drug loaded therein.

### TECHNICAL FIELD

The present invention relates to a bone graft composition and a preparation method thereof, and more particularly, a bone graft composition provided in the form of a putty formulation by mixing calcium phosphate compound particles with hydrogel, having excellent physical properties, which is easy to inject, and which maintains its structure even in an in vivo environment after implantation, thereby enabling sustained release of a drug loaded therein.

### BACKGROUND ART

Bone graft biomaterials developed in the initial stages depended on a characteristic that they are inert in vivo, but the use thereof was significantly limited due to infection and inflammatory reaction which occur in the surrounding tissue after implantation. Since then, with the rapid development of biomaterial technologies based on metals, ceramics, and polymer materials, materials were designed and developed that are biocompatible rather than bioinert, leading to the development of bioactive scaffolds for bone tissue regeneration, which vary depending on the site and purpose of use. It is required that such bioactive scaffolds for bone tissue regeneration have different physical properties depending on the location of graft placement, that they not be toxic to the surrounding tissue, and that they have relatively high mechanical properties compared to other artificial organs. Such bioactive scaffolds for bone tissue regeneration have been marketed and developed as various biomaterials depending on the properties of the raw materials and the intended use thereof.

All materials that are to be grafted into the human body, particularly polymer materials for bone tissue regeneration, should have good processability and moldability or have good in situ polymerization properties so as to be suited to wounds. These materials are required to provide a suitable environment for the adhesion, growth, and differentiation of cells, and degradation products thereof are also required to be biocompatible. In particular, if compressive strength and yield value of a bone graft material are too low, it will be difficult to maintain the abilities of the bone graft material to fix its location and keep its external shape in the closure or implant placement stage after injection or dense filling of the bone graft material. In addition, if adhesiveness of a bone graft material is too high, it will easily stick to a surgical tool during surgery, and thus it will be difficult to easily fill in bone defects, resulting in a decrease in workability.

Accordingly, there is a need for the development of a bone graft composition that has biocompatibility and physical properties suitable for grafting in bone defects and has the property of maintaining the formulation during a specific period after implantation.

Meanwhile, hydroxyapatite, which is a calcium phosphate-based ceramic, is a component found in teeth and bones. Since hydroxyapatite has excellent biocompatibility, it has attracted attention as a graft material for implantation into the body, such as fillers or artificial implants to replace damaged bones. However, since hydroxyapatite itself has a ceramic particle-type formulation, molding thereof is impossible, and thus it is difficult to apply to a narrow site. There is also a disadvantage in that it is difficult to fill hydroxyapatite in a specialized material intervertebral body fusion cage used in lumbar interbody fusion surgery.

The present inventors have also tried to provide a bone graft composition including a calcium phosphate compound, which is injectable into the body, as disclosed in a prior patent (Korean Patent No. 10-1443814). However, the structure of the composition is easily disintegrated in an in vivo environment where blood flow exists, resulting in release of all drugs or physiologically active substances loaded therein within a short time. Thus, it is difficult to anticipate their continuous effects.

Accordingly, the present inventors have made intensive efforts to develop a formulation in which the above-described disadvantages of the existing bone graft composition including a calcium phosphate compound are improved, and as a result, they found that when the content of the calcium phosphate compound to be used is increased while controlling a size distribution of the particles to be used, its structure is maintained even in an environment in contact with a fluid, like that in a living body, and thus the sustained release of a physiologically active substance or a drug loaded therein is possible, thereby completing the present invention.

### DISCLOSURE

### Technical Problem

An object of the present invention is to provide an injectable bone graft composition including more than 55 wt % (% by weight) and 80 wt % or less of calcium phosphate compound particles; and 20 wt % or more and less than 45 wt % of biodegradable hydrogel.

Another object of the present invention is to provide a kit for bone implantation, the kit including the bone graft composition and an injection tool.

A further object of the present invention is to provide use of composition including more than 55 wt % (% by weight) and 80 wt % or less of calcium phosphate compound particles; and 20 wt % or more and less than 45 wt % of biodegradable hydrogel for bone graft.

### Technical Solution

Each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description below.

Further, those skilled in the art will recognize, or be able to ascertain using no more than routine experimentation, many equivalents to the specific embodiments of the invention described herein. Further, these equivalents should be interpreted to fall within the scope of the present invention.

In addition, throughout this specification, when a part is referred to as “including” an element, it will be understood that other elements may be further included rather than other elements being excluded unless content to the contrary is specially described.

Hereinafter, the present invention will be described in detail.

The present invention provides an injectable bone graft composition including more than 55 wt % and 80 wt % or less of calcium phosphate compound particles; and 20 wt % or more and less than 45 wt % of biodegradable hydrogel.

Since the composition of the present invention has a predetermined fluidity, it is advantageous when injected into irregular defects, while having excellent physical properties, and thus its microstructure may be maintained in an in vivo environment after implantation, and the composition may be usefully applied as a bone graft material.

In particular, when the composition is prepared by mixing calcium phosphate compound particles with biodegradable hydrogel, a mixture of microparticles and macroparticles is used as the calcium phosphate compound included in the composition. Therefore, its content is increased at a high ratio of 70% or more, thereby providing a graft material having remarkably improved strength.

Further, since the composition includes hydrogel containing a biodegradable polymer, it may be biodegraded over time after implantation. In addition, since hydrogel containing a predetermined amount of a poloxamer and HPMC in combination is used, a compressive strength and a yield value are high, thereby providing a bone graft composition with excellent volume retention in the body temperature range after bone implantation. In addition, since the composition includes the hydrogel and the calcium phosphate compound particles at a suitable mixing ratio, it may have a formulation such as a putty type resulting from the agglomeration of the hydrogel and the calcium phosphate compound particles. At the same time, the composition has low adhesiveness, and thus it does not stick to a surgical tool during surgery, and it does not stick to a surgical tool when it is filled in bone defects, suggesting that it has an advantage of excellent workability.

In particular, in the case of the existing composition containing only β-TCP fine particles (or microspheres) as the calcium phosphate compound in addition to the hydrogel, it is difficult to prepare a formulation itself when the content of β-TCP fine particles (or microspheres) increases over a predetermined range. When a composition is prepared using the maximum amount capable of preparing the formulation, the desired strength may not be achieved, and thus there is a problem in that its structure may not be maintained in the in vivo environment when transplanted into a bone defect.

As used herein, the term “bone graft composition” refers to a composition for use as bone defect replacement that is grafted in bone defects to fill the bone defects. Specifically, the bone graft composition in the present invention means a synthetic bone graft material (alloplastic) composition based on a calcium phosphate compound.

The bone graft composition of the present invention is mainly composed of two components of calcium phosphate compound particles and hydrogel.

First, the calcium phosphate compound particles are similar to natural bone and functions to induce osteoconduction and bone growth.

As used herein, the term “calcium phosphate compound” may refer to a compound including phosphoric acid and calcium. Specifically, the calcium phosphate compound may be any one or a combination of two or more selected from the group consisting of hydroxyapatite, tricalcium phosphate (TCP, Ca₃(PO₄)₂), tetracalcium phosphate (Ca₄(PO₄)₂O), brushite (CaHPO_{4.2}H₂O), dicalcium diphosphate (Ca₂P₂O₇), calcium tripolyphosphate (Ca₅(P₃O₁₀)₂), Mg-containing apatite, Mg-containing TCP, Sr-containing apatite, and fluorapatite. More specifically, tricalcium phosphate, for example, β-TCP and hydroxyapatite, may be used in a mixture, but the calcium phosphate compound is not limited thereto.

In this regard, as the calcium phosphate compound particles, a mixture of porous particles of 45 μm to 100 μm and 200 μm to 6000 μm in mean diameter may be used. Generally, considering that the particle size is not defined by a single size, but the diameters of particles having a certain distribution are statistically averaged, the calcium phosphate compound particles may be a mixture of particles having a size of 45 μm to 6000 μm in diameter, but are not limited thereto.

For example, in a specific embodiment of the present invention, a mixture of microspherical β-TCP and hydroxyapatite macroparticles was used.

In this regard, the β-TCP may be in the form of microspheres, but is not limited thereto. For example, the β-TCP may be obtained by spray-drying β-TCP powder, sintering the dried powder at a temperature of 1050° C. to 1250° C., and classifying the sintered powder in the range of 45 μm to 75 μm. More specifically, the β-TCP may form a spherical shape during spray-drying of the β-TCP powder, and porosity of the powder may be increased by sintering the spherical β-TCP powder at a temperature of 1050° C. to 1250° C. In order to obtain a more uniform bone graft composition, the sintered β-TCP powder may be classified in the range of 45 μm to 75 μm. At this time, the sintering may be performed for 1 hour to 3 hours, and most preferably, for 2 hours.

As a result, the β-TCP particles finally obtained as described above may be microspherical particles having a diameter of 45 μm to 75 μm. Moreover, the final β-TCP particles may have porosity of 60% or higher as a result of performing the spray-drying and sintering processes as described above.

Meanwhile, the hydroxyapatite may be a granule having a broad size distribution of several tens of μm to several mm, but is not limited thereto.

Further, the calcium phosphate compound particles may be porous particles having a three-dimensional pore connectivity of 90% or more and/or porosity of 60% or more. For example, by using the porous calcium phosphate compound particles, a drug and/or a bone morphogenetic protein may be loaded in the pores thereof, as needed, thereby exhibiting two or more effects at the same time or achieving a synergistic therapeutic effect. Furthermore, since the porous structure facilitates penetration of newly formed tissues, it thereby promotes tissue regeneration.

The hydrogel which is the second component of the bone graft composition of the present invention is a gel formed by dispersing a polymer having a sol-gel transition property in water, and is a means that agglomerates the calcium phosphate compound particles to form a formulation suitable for bone grafting.

The hydrogel may include one or more selected from the group consisting of a poloxamer, collagen, hyaluronic acid, gelatin, a PEG/PPG/PEG block copolymer, and cellulose. The hydrogel, which is a material having a non-crosslinked structure, may be a material without a swelling property, and may be a material that decomposes within several months. In this regard, the hydrogel may include the above-described components at a concentration of 15 wt % to 35 wt %, but is not limited thereto. If the concentration of the hydrogel is less than 15 wt %, it may be difficult to have sufficient strength, and if the concentration is more than 35 wt %, its adhesiveness is high, and thus a large amount thereof may remain in a container for manufacturing and/or storage or in a tool for transport and/or injection.

In the present invention, the hydrogel may further include 0.5 parts by weight to 2 parts by weight of hydroxypropyl methylcellulose (HPMC), based on 100 parts by weight of the hydrogel.

In the present invention, a poloxamer and hydroxypropyl methylcellulose (HPMC) may be used as polymers which are biodegradable, which have a sol-gel transition temperature lower than the body temperature, and which may maintain the gel state in the body temperature range, in order to provide a bone graft composition that has excellent biocompatibility and to have an excellent ability to maintain its formulation after implantation.

As used herein, the term “poloxamer” refers to a triblock copolymer (PEO-PPO-PEO) having two polyethylene glycol (PEG) chains bonded to a central chain of polypropylene glycol (PPG). Generally, a ratio of PEG/PPG in a poloxamer may vary in the range from 1:9 to 8:2. A molecular weight of a poloxamer may be in a wide range from 1,100 g/mol to 14,000 g/mol. A poloxamer is a temperature-sensitive polymer. In the present invention, the poloxamer functions to impart injectability and moldability to the bone graft composition and to enable the bone graft material to be degraded rapidly after filling in bone defects so as to allow only the calcium phosphate-based bone graft material component to remain. In order to maintain the ease of injection and moldability in the room temperature range and formulation stability during storage and transport at room temperature, a high-molecular-weight poloxamer having a relatively low sol-gel transition temperature and high viscosity is preferably used. Preferably, a poloxamer that has a sol-gel transition temperature of 4° C. to 35° C. so as to be able to maintain the gel state at the body temperature of about 37° C. may be used in the present invention. Specifically, poloxamer 407, having an excellent ability to maintain the gel state at the body temperature of about 37° C., may be most preferably used in the present invention.

As used herein, the term “hydroxypropyl methylcellulose (HPMC)” refers to a semi-synthetic, inert, viscoelastic polymer, also called hypromellose. In the present invention, HPMC functions to improve the elasticity of the hydrogel. In particular, as the viscoelasticity of the hydrogel increases, the ability to fix the location of the bone graft material when filling in bone defects becomes better so that the leakage of the bone graft material to the outside may be advantageously minimized.

The viscosity of HPMC may be preferably 1,000 cps to 100,000 cps, and most preferably 100,000 cps. HPMC is added in a trace amount in order to induce high viscosity and high elasticity. As the viscosity thereof increases, the ability to fix the location of the bone graft material and the density of filling of the bone graft material may be increased, and the adhesion of the bone graft material to a surgical tool and gloves may be minimized Thus, it is most preferable to use HPMC having a viscosity of 100,000 cps, which shows the highest viscosity when being added in a trace amount.

In the composition of the present invention, the hydrogel including HPMC in addition to a specific amount of a poloxamer is used, thereby providing a bone graft composition that is better in terms of compressive strength, yield value, and adhesiveness as compared with a hydrogel including a poloxamer alone or a hydrogel outside of the above-described ranges.

Particularly, the bone graft composition of the present invention may have a yield value ranging from 1500 g/cm² to 4000 g/cm². When the composition of the present invention has a yield value within the above-described range, it will exhibit excellent viscoelasticity, and thus may be easily filled in bone defects during grafting, suggesting that it shows physical properties suitable for use as a graft material.

As used herein, the term “compressive strength” is used interchangeably with “strength”, and means the strength at which the external shape of the bone graft composition is changed by an external force. The term “yield value” is a physical property value related to the elasticity of a finished product, and means the maximum strength at which the composition is not deformed by an external force. Thus, as the compressive strength and yield value increase, it is possible to maintain the abilities of the composition to fix its location and maintain its external shape in the closure or the implant placement stage after injection or dense filling of the bone graft material.

As used herein, the term “adhesiveness” means the property of adhering to stainless steel. It is a force acting in a direction opposite to that of compressive strength, and (−) means only direction. The higher absolute value of the adhesiveness means that a greater force is required to detach the bone graft composition formulation that stuck to stainless steel, and it may also mean the degree of adhesion of the bone graft composition formulation not only to a surgical tool made of stainless steel, but also to gloves made of resin.

Compressive strength, yield value, and adhesiveness, which are physical strengths that are measured in the present invention, may be measured using a common rheometer and/or UTM (universal testing machine).

The bone graft composition of the present invention includes a hydrogel filled between calcium phosphate compound particles close to each other. After implantation of the bone graft composition into bone defects, the hydrogel is degraded and released, the calcium phosphate compound particles are maintained in the close state, and bone grows into the space between the calcium phosphate compound particles after release of the hydrogel. Therefore, it is necessary to have the ability to maintain its shape for a predetermined period of time.

The bone graft composition of the present invention may further include a physiologically active substance. The physiologically active substance may be loaded in pores of the porous calcium phosphate compound particles included in the composition. The physiologically active substance may be one or more selected from the group consisting of bone morphogenetic proteins (BMPs), bone morphogenetic peptides, extracellular matrix proteins, and tissue growth factors.

For example, the bone morphogenetic proteins may include BMP-2, BMP-3, BMP-3b, BMP-4, BMP-5, BMP-6, BMP-7, BMP-8, BMP-9, BMP-10, BMP-11, BMP-12, BMP-13, BMP-14, BMP-15, BMP-16, BMP-17, BMP-18, or a combination thereof, but are not limited thereto.

For example, the tissue growth factors may include VEGF, FGF-2, IGF-1, TGF-β, or the like, but are not limited thereto.

For example, the bone graft composition of the present invention may be used in bone grafting, maxillary sinus lifting, lumbar interbody fusion, cervical interbody fusion, or upper & lower extremity fracture fusion, but is not limited thereto. For example, the bone graft composition may be used in lateral lumbar interbody fusion (LLIF), oblique lumbar interbody fusion (OLIF), or anterior lumbar interbody fusion (ALIF), but is not limited thereto. In particular, the bone graft composition of the present invention is a putty formulation which may be injected regardless of the shape of the site to be transplanted, and has excellent strength, and thus may be usefully applied to the bone damage site having an unspecified shape.

The composition of the present invention may be provided in a putty formulation.

Another aspect of the present invention provides a kit for bone implantation, the kit including the bone graft composition and an injection tool.

The kit of the present invention may be a bone graft material of a putty formulation. Such a graft material of the putty formulation may be injected into a desired site by putting it in an injection tool such as a syringe, tube, etc. After injection, the bone graft material may be packed by a surgical tool so that it is densely filled into bone defects due to a viscoelastic property of the product.

To this end, the kit of the present invention may further include an injection tool, wherein the injection tool may include a mixing syringe or a vial transport device, but is not limited thereto.

As described above, the kit for bone implantation of the present invention may be used in bone grafting, maxillary sinus lifting, lumbar interbody fusion, cervical interbody fusion, or upper & lower extremity fracture fusion, e.g., lateral lumbar interbody fusion (LLIF), oblique lumbar interbody fusion (OLIF), or anterior lumbar interbody fusion (ALIF), but is not limited thereto.

A further aspect of the present invention provides use of composition including more than 55 wt % (% by weight) and 80 wt % or less of calcium phosphate compound particles; and 20 wt % or more and less than 45 wt % of biodegradable hydrogel for bone graft.

### Advantageous Effects

The composition of the present invention is designed to compensate for the disadvantages of the existing formulation, for which it is difficult to improve physical properties, by using a microparticle-type calcium phosphate-based compound and increasing the content thereof. When a bone graft material is prepared by mixing the calcium phosphate-based compound with hydrogel, a mixture of microparticles and macroparticles is used as the calcium phosphate-based compound to increase the content of the calcium phosphate-based compound by 70% or more based on the total composition. Thus, the composition may maintain its shape for a long period of time under in vivo mimetic conditions, and enables sustained release of a drug loaded therein, and therefore, it may be usefully applied as a graft material for regeneration of injured bone tissues by loading physiologically active substances such as bone morphogenetic proteins, etc.

### DESCRIPTION OF DRAWINGS

FIG. 1 shows actual appearances of various sizes (r) of calcium phosphate-based compound particles;

FIG. 2 shows appearances of compositions prepared by mixing various sizes of calcium phosphate-based compound particles with hydrogel at predetermined ratios;

FIG. 3 shows an exemplary formulation of a composition according to one exemplary embodiment of the present invention, wherein the left shows the use of the composition filled in a putty-type syringe which is used in dentistry, and the right shows the use of the composition filled in a case which is used in spine fusion surgery;

FIG. 4 shows elasticity and/or texture of the composition according to one exemplary embodiment of the present invention;

FIG. 5 shows shape retention ability of bone graft materials under in vivo mimetic conditions, the bone graft materials composed of the compositions which were prepared by mixing various sizes of calcium phosphate-based compounds with hydrogel at predetermined ratios;

FIG. 6 shows sustained drug release of bone graft materials under in vivo mimetic conditions, the bone graft materials composed of the compositions which were prepared by mixing various sizes of calcium phosphate-based compounds with hydrogel at predetermined ratios;

FIG. 7 shows shear strain vs. shear stress of the bone graft material prepared according to one exemplary embodiment of the present invention in a fluid environment (black solid line), wherein as a control (blue solid line), a bone graft composition of Comparative Example 6 was used; and

FIG. 8 shows changes in compressive strength according to the sizes of the calcium phosphate compound particles of the bone graft materials prepared according to one exemplary embodiment of the present invention.

### MODE FOR INVENTION

Hereinafter, the configuration and effects of the present invention will be described in more detail with reference to exemplary embodiments. However, these exemplary embodiments are for illustrative purposes only, and the scope of the present invention is not intended to be limited by these exemplary embodiments.

### PREPARATION EXAMPLE 1: Preparation of calcium phosphate Compound Particle Powder 1

Pure β-TCP powder (Cerectron Co., Korea) was spray-dried to prepare a spherical shape. Then, the spherical β-TCP powder was sintered at 1050° C., and the sintered particles were classified in the range of 45 μm to 75 μm.

### PREPARATION EXAMPLE 2: Preparation of calcium phosphate Compound Particle Powder 2

Calcium phosphate compound particles having a distribution in the range of 200 μm to 6,000 μm were prepared with reference to the method disclosed in Korean Patent No. 10-0401941.

### EXAMPLE 1: Preparation of High-Elasticity calcium phosphate-Based Bone Graft Material

First, HPMC and poloxamer 407 were mixed using a high-speed vacuum mixer to produce hydrogel, and then the β-TCP powder prepared according to Preparation Example 1 was uniformly mixed therewith to obtain a hydrogel complex.

Subsequently, the prepared hydrogel complex was mixed with the hydroxyapatite ceramic granules having a size of 0.6 mm to 6 mm prepared according to Preparation Example 2 to prepare a bone graft material of a putty formulation. The mixing was performed using a specialized mixing syringe so that the hydroxyapatite granules were pulverized.

### COMPARATIVE EXAMPLES 1 to 4: Various Sizes of Calcium Phosphate-Based Compound Particles

Particle-type calcium phosphate-based compounds having a size in the range of less than 100 μm, 600 μm to less than 1,000 μm, 1,000 μm to less than 3,000 μm, and 3,000 μm to 6,000 μm were prepared using the samples of Comparative Examples 1 to 4, respectively.

### COMPARATIVE EXAMPLES 5 to 7: Bone Graft Composition Including Controlled Contents of calcium phosphate-based Compound Microparticles and Hydrogel

Calcium phosphate-based compound particles having a size of less than 100 μm and hydrogel were mixed at a weight ratio of 30:70, 50:50, and 70:30 to prepare bone graft compositions of Comparative Examples 5 to 7, respectively.

### COMPARATIVE EXAMPLE 8: Bone Graft Composition Including Controlled Contents of Calcium Phosphate-Based Compound Macroparticles and Hydrogel

Calcium phosphate-based compound particles having a size of 1,000 μm to less than 3,000 μm and hydrogel were mixed at a weight ratio of 50:50 to prepare a bone graft composition of Comparative Example 8.

### EXPERIMENTAL EXAMPLE 1: Appearance and Physical Properties of Bone Graft Composition

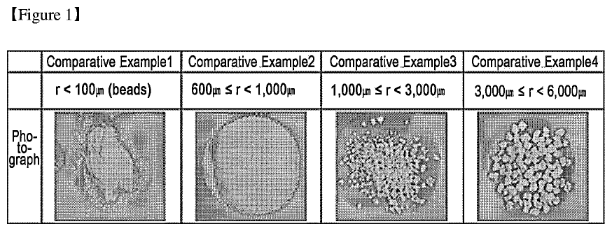

The shapes of the calcium phosphate-based compound particles of Comparative Examples 1 to 4 were observed with the unaided eye and photographed, and are shown in FIG. 1. As shown in FIG. 1, when the calcium phosphate-based compounds were used alone, it was difficult to process the compounds into a desired shape because they were composed of particles, and thus their use as a bone graft material was very limited.

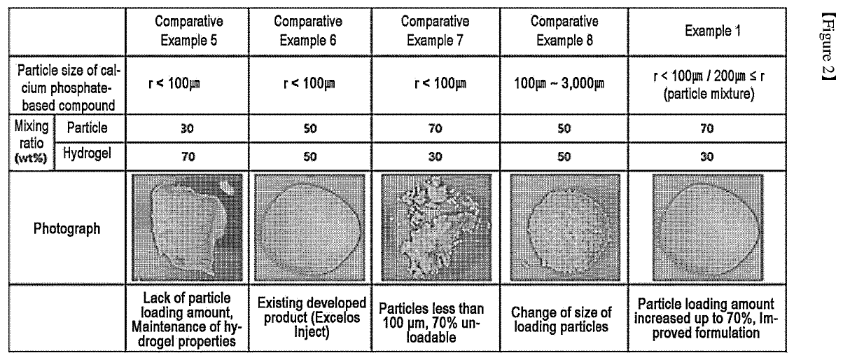

Further, appearance and features of the bone graft compositions of Comparative Examples 5 to 8 and Example 1 are shown in FIG. 2. As shown in FIG. 2, the bone graft compositions composed of calcium phosphate-based compound particles having a size of less than 100 μm and hydrogel showed the difference in appearance according to the composition ratio of these components, which was visible to the unaided eye. When the content of the calcium phosphate-based compound was as low as 30% (Comparative Example 5), the particle loading amount was insufficient, and thus the physical properties closer to the hydrogel were maintained. However, when the content of the calcium phosphate-based compound was 50% (Comparative Example 6, Excelos Inject), particles agglomerated well with the hydrogel to form and maintain a clay-like shape, similar to Example 1, when examined with the unaided eye. Meanwhile, in the composition (Comparative Example 8), which was prepared by mixing calcium phosphate-based compound particles having a size of 1,000 μm to less than 3,000 μm and hydrogel at a weight ratio of 50:50, although the particles agglomerated into a single mass, a structure with a rough surface was formed due to individual particles. Furthermore, as in the composition of Example 1, when the content of the calcium phosphate-based compound was increased by 70% (Comparative Example 7), only microparticles with a size of less than 100 μm could not agglomerate into a single mass and crumbled due to the excessive loading amount of particles. Meanwhile, as in Comparative Example 7, even though the content of the calcium phosphate-based compound was as high as 70%, when macroparticles having a size of 200 μm or more were further included in addition to calcium phosphate-based compound microparticles having a size of less than 100 μm, they were found to aggregate with the hydrogel to form a single mass, as in Comparative Example 6. This suggests that a bone graft composition having a higher content of calcium phosphate compound may be provided by using a mixture of microparticles and macroparticles.

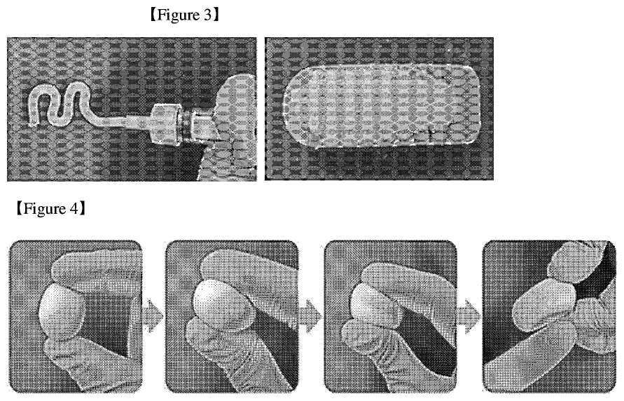

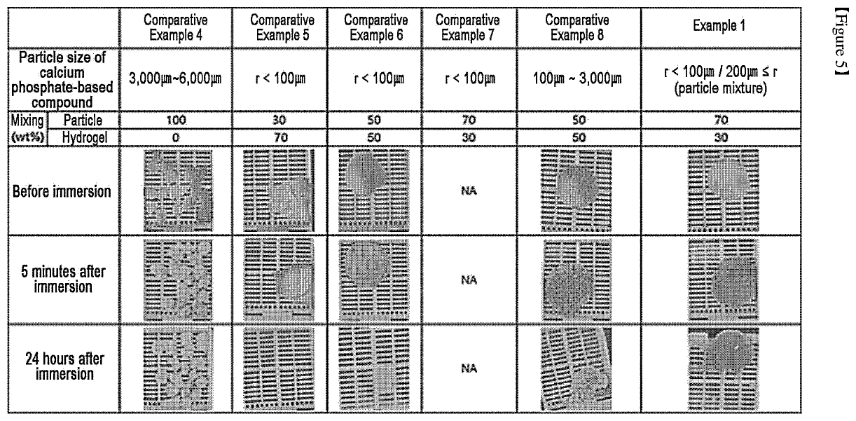

As described, the bone graft composition of Example 1, which was prepared by including the high 70% content of the calcium phosphate-based compound, was formulated into various preparations, and the clinical applicability thereof was tested. The test results are shown in FIGS. 3 and 4. As shown in FIG. 3, the bone graft composition of Example 1 could be injected using a dental putty-type syringe, and it was easily filled in a cage used for spinal fusion. Further, as shown in FIG. 4, when the shape was deformed by pressing it even with fingers, the composition could be easily deformed into a desired shape and did not stick to the fingers, and it was advantageous in controlling, with no concern about loss. As described above, since the bone graft composition of the present invention has enough fluidity to be injected using a syringe, it may be directly injected into a defect where it is difficult to accomplish a desired shape. It is also easy to obtain a desired shape by hand or using a predetermined cast, and the corresponding shape may be maintained. Thus, it may be used for bone regeneration.

### EXPERIMENTAL EXAMPLE 2: Shape Retention Ability and Sustained Drug Release Under In Vivo Mimetic Conditions

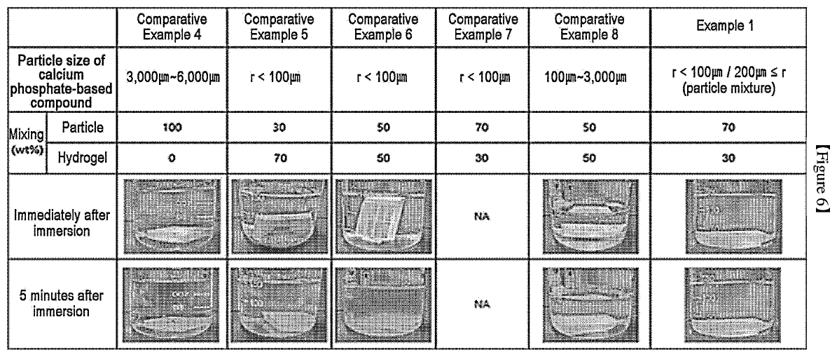

To examine properties of the bone graft materials under in vivo mimetic conditions, each of the compositions of Comparative Examples 4 to 8 and Example 1 was put in a cage and immersed in physiological saline at 37° C. After 5 minutes and 24 hours of immersion, their shape retention was examined. The results are shown in FIG. 5. Furthermore, in order to examine the release patterns of the bone graft materials when a drug was loaded therein, a red dye was loaded instead of the drug so that each sample was visually identified and then treated as described above. The color of the solution was examined before immersing in physiological saline and after 5 minutes of immersion, and the results are shown in FIG. 6.

As shown in FIG. 5, when the macroparticle-type calcium phosphate-based compound was used alone (Comparative Example 4), even at 24 hours after being immersed in physiological saline similar to the body temperature, no change in the shape was visually observed. When the content of the calcium phosphate-based compound was as low as 30% (Comparative Example 5), the shape was actually maintained until 5 minutes after being immersed in physiological saline, but after 24 hours, it was completely decomposed, and the shape could not be identified. This indicates that no strong physical bond was formed between the calcium phosphate-based compound particles and the hydrogel. When the content of the calcium phosphate-based compound was 50% (Comparative Example 6, Excelos Inject), an almost intact shape was maintained until 5 minutes after being immersed in physiological saline. However, after 24 hours, most of the particles were decomposed and only partly remained. This indicates that the particles still failed to form a bond strong enough to withstand the in vivo mimetic conditions. Meanwhile, when the content of the calcium phosphate-based compound was as high as 70% (Comparative Example 7), the desired shape could not be obtained because the particles were not sufficiently combined as they are, and thus additional experiments were not possible, as confirmed in Experimental Example 1. Furthermore, the composition including 50% of the calcium phosphate-based compound macroparticles (Comparative Example 8) exhibited excellent shape retention ability, as compared with the composition of Comparative Example 6 including the same amount of the microparticles, but a significant portion thereof was decomposed. In contrast, the composition of Example 1, which was prepared by including 70% of both the microparticles and the macroparticles, maintained the existing shape without structural decomposition even after 24 hours. This suggests that the graft material composed of the composition of Example 1 is a material suitable for use as a bone graft material, which is able to maintain its structure even in a practical surgical environment such as bone implantation and/or spinal fusion.

As shown in FIG. 6, the graft materials of Comparative Examples 4 to 6 and 8, excluding Comparative Example 7, in which experimentation was impossible, began to release the dye which was clear enough to be identified with the unaided eye, immediately after being immersed in physiological saline, and after 5 minutes, most dye was released. In detail, as compared with Comparative Example 4, in which the calcium phosphate-based compound particles were used alone, when a predetermined amount of hydrogel was included, the degree was slightly reduced. In particular, in the composition of Comparative Example 5 including the hydrogel at as high as 70%, initial release was considerably inhibited, indicating that diffusion of the dye was physically inhibited by the hydrogel. In contrast, the graft material of Example 1 released a small amount of dye, but the degree was insignificant, and the released amount was significantly smaller than the results of other Comparative Examples. This result was also confirmed from FIG. 5, showing that the color of the graft material became pale. Taken together, in the composition of Example 1, even though the content of hydrogel was slightly low at 30%, the release of the dye was overall inhibited, suggesting that when a drug is loaded in the composition of Example 1, sustained release thereof may be achieved.

### EXPERIMENTAL EXAMPLE 3: Comparison of Yield Stress in Fluid

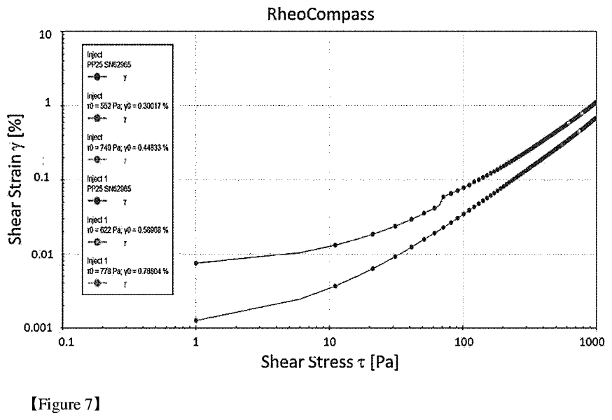

The same force (shear stress) was applied using a rheometer to the graft material composed of the composition of Comparative Example 6, which is a commercially available product, and the graft material composed of the composition of Example 1, in which calcium phosphate-based compound macroparticles were additionally included to increase the content thereof. At this time, the shear strain of each formulation was measured and shown in FIG. 7. As shown in FIG. 7, it was confirmed that when the same force was applied, the deformation of the control group was more severe than that of the specimen of Example 1. This indicates that the formulation of Example 1 is able to maintain its shape without structural decomposition, when in contact with water or blood flow in the in vivo environment in which it is implanted, that is, when a physical force is applied, and therefore, a drug, e.g., BMP-2, loaded therein may be sustained-released.

### EXPERIMENTAL EXAMPLE 4: Comparison of Strength According to Particle Size of calcium phosphate Compound

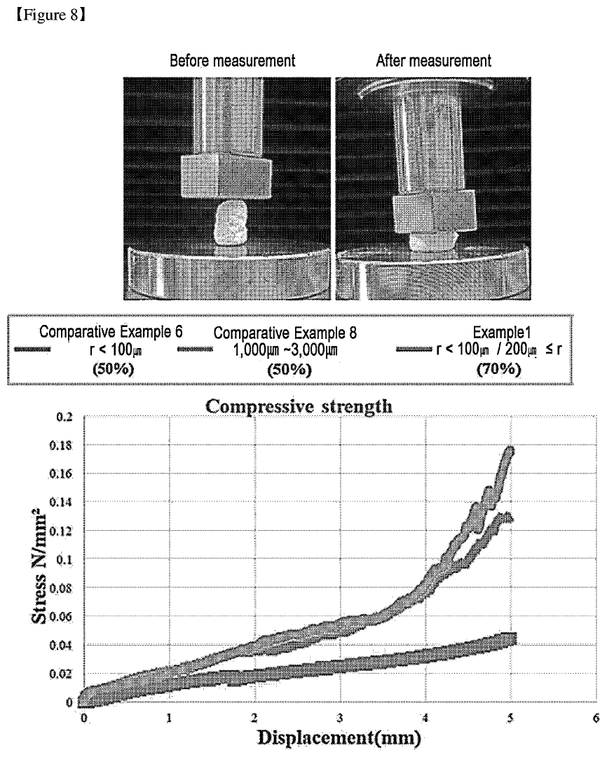

In order to examine changes in the strength of the composition of Example 1 in which the content of calcium phosphate-based compound was increased by additionally including macroparticles in addition to calcium phosphate-based compound microparticles, the composition of Example 1 in which the content of the calcium phosphate-based compound was increased to 70% by including both microparticles and macroparticles, the composition of Comparative Example 6 in which the content of the calcium phosphate-based compound was 50% by including only microparticles, and the composition of Comparative Example 8 in which the content of the calcium phosphate-based compound was 50% by including only macroparticles were measured for compressive strength, and the results are shown in FIG. 8. In detail, specimens of 8 mm×10 mm in size were prepared by using each composition, and deformation under compression was measured, as shown at the top of FIG. 8. As shown in FIG. 8, when Comparative Examples 6 and 8 were compared with each other, Comparative Example 8 showed remarkably improved strength, in which the composition had the same content, but the particle had the larger size. In contrast, the composition of Example 1 including both microparticles and macroparticles showed improved strength due to the increased content thereof, because even though microparticles were included, they were mixed with the macroparticles, as compared to that of Comparative Example 8.

Based on the above description, it will be understood by those skilled in the art that the present disclosure may be implemented in a different specific form without changing the technical spirit or essential characteristics thereof. Therefore, it should be understood that the above embodiment is not limitative, but illustrative in all aspects. The scope of the disclosure is defined by the appended claims rather than by the description preceding them, and therefore all changes and modifications that fall within metes and bounds of the claims or equivalents of such metes and bounds are therefore intended to be embraced by the claims.

1. An injectable bone graft composition comprising:
more than 55 wt % (% by weight) and 80 wt % or less of calcium phosphate compound particles; and
20 wt % or more and less than 45 wt % of biodegradable hydrogel.

2. The injectable bone graft composition of claim 1, wherein the calcium phosphate compound is any one or a combination of two or more selected from the group consisting of hydroxyapatite, tricalcium phosphate (TCP, Ca₃(PO₄)₂), tetracalcium phosphate (Ca₄(PO₄)₂O), brushite (CaHPO_{4.2}H₂O), dicalcium diphosphate (Ca₂P₂O₇), calcium tripolyphosphate (Ca₅(P₃O₁₀)₂), Mg-containing apatite, Mg-containing TCP, Sr-containing apatite, and fluorapatite.

3. The injectable bone graft composition of claim 1, wherein the calcium phosphate compound particles are porous particles having a size of 45 μm to 100 μm and 200 μm to 6,000 μm in mean diameter.

4. The injectable bone graft composition of claim 3, wherein the porous particles have porosity of 60 vol % (% by volume) or more.

5. The injectable bone graft composition of claim 1, wherein the hydrogel includes one or more selected from the group consisting of a poloxamer, collagen, hyaluronic acid, gelatin, a PEG/PPG/PEG block copolymer, and cellulose.

6. The injectable bone graft composition of claim 5, wherein the hydrogel is a material having a non-crosslinked structure without a swelling property.

7. The injectable bone graft composition of claim 1, further comprising a physiologically active substance.

8. The injectable bone graft composition of claim 7, wherein the physiologically active substance is one or more selected from the group consisting of bone morphogenetic proteins, bone morphogenetic peptides, extracellular matrix proteins, and tissue growth factors.

9. The injectable bone graft composition of claim 1, wherein the injectable bone graft composition is used in bone grafting, maxillary sinus lifting, lumbar interbody fusion, cervical interbody fusion, or upper & lower extremity fracture fusion.

10. The injectable bone graft composition of claim 1, wherein the injectable bone graft composition is a putty formulation.

11. A kit for bone implantation, the kit comprising the bone graft composition of claim 1 and an injection tool.

12. The kit of claim 11, wherein the injection tool includes a mixing syringe or a vial transport device.

13. The kit of claim 11, wherein the bone graft composition further comprises a physiologically active substance.

14. The kit of claim 13, wherein the physiologically active substance is one or more selected from the group consisting of bone morphogenetic proteins, bone morphogenetic peptides, extracellular matrix proteins, and tissue growth factors.

15. The kit of claim 11, wherein the bone graft composition is used in bone grafting, maxillary sinus lifting, lumbar interbody fusion, cervical interbody fusion, or upper & lower extremity fracture fusion.
